(19) United States
(12) Patent Application Publication
(10) Pub. No.: US 2024/0274243 A1
(43) Pub. Date: Aug. 15, 2024

(54) METHOD AND SYSTEM TO PREDICT AT LEAST ONE PHYSICO-CHEMICAL AND/OR ODOR PROPERTY VALUE FOR A CHEMICAL STRUCTURE OR COMPOSITION

(71) Applicant: Firmenich SA, Satigny (CH)

(72) Inventors: Guillaume GODIN, Satigny (CH); Ruud VAN DEURSEN, Satigny (CH); Florian RAVASI, Morges (CH)

(21) Appl. No.: 18/534,565

(22) Filed: Dec. 8, 2023

(30) Foreign Application Priority Data
Dec. 8, 2022 (EP) 22212124.6

(51) Int. Cl. G16C 20/30; G16C 20/70

(52) U.S. Cl. CPC G16C 20/30; G16C 20/70

(57) ABSTRACT

The method (100) to predict physico-chemical and/or odor properties value for chemical structures or compositions comprises the steps of:
defining (105) a representation of a chemical structure or composition,
executing (110) upon the representation defined, an end-to-end trained ensemble neural network or multi-branch neural network model to predict a physico-chemical and/or odor property value,
providing (115) the physico-chemical and/or odor property value,
the method further comprising:
   providing (120) exemplar data to an end-to-end ensemble neural network or multi-branch neural network device comprising:
      several neural network sub-devices configured to independent predictions,
      a layer to output at least one value of the distribution of independent predictions and
      said layer comprising a sampling device configured to output random values,
   operating (125) the end-to-end ensemble neural network or multi-branch neural network device and
   obtaining (130) the trained ensemble neural network or multi-branch neural network model.

### TECHNICAL FIELD OF THE INVENTION

The present invention aims at a method to predict at least one physico-chemical and/or odor property value for a chemical structure or composition, a system to predict at least one physico-chemical and/or odor property value for a chemical structure or composition and a method to efficiently assemble chemical structures or compositions.

It applies, in particular, to the industry of flavors and fragrances.

### BACKGROUND OF THE INVENTION

In scientific experiments, measurements 305 stored in databases, such as shown in FIG. 3, vary due to the environment of said experiment. One typically needs to use an ideal environment such as the international space station to get stable conditions. Even in near perfect environmental conditions, instruments and sample preparation by technicians may slightly vary from one to another. Merging experimental data from different sources can be difficult because of changing experimental variations depending on measurement conditions used. Statistical methods to homogenize experimental data have been developed to reduce those variations with moderate to good success. In the field of machine learning, such variations may exist between training and test sets as well as between the known and future data.

Another well-known issue of machine learning models is the number of hyperparameters in the model, which may significantly influence a model's ability to overfit the training data 310, such as shown in FIG. 3. The amount of data available may be insufficient to train the number of hyperparameters in a neural network. Therefore, it is best to consider the minimum need to extract a meaningful digital data representation for the data. The latter can be achieved by favoring layers with a lower number of parameters. An example of such an attempt is replacing dense layers by compact convolution layers. Convolution layers are particularly dedicated to extract from data localized features like images. However, even by using compact and efficient convolutional layers, networks with billions of parameters may still be generated. Trending mega-models show that such models can only be trained with very large datasets, which are, for instance, available for images, literature, and music. On the contrary, to avoid costly experiments as well as to stop animal testing, scientific fields like chemistry and biology frequently have limited datasets with some hundreds to thousands of data points. In such domains, one should thus aim at a reduction on the number of parameters to match the size of the data.

One way to compensate for the large size of the networks is by data augmentation. Indeed, increasing performances linked to an increasing augmentation rate indicates that more data is needed for the selected size of the network, suggesting that the size of the network can be possibly reduced (Tetko, I. V., Karpov, P., Van Deursen, R. et al. State-of-the-art augmented NLP transformer models for direct and single-step retrosynthesis. Nat Commun 11, 5575 (2020)). Simultaneously, augmentation can be used to identify if a network is critically parametrized, i.e., the point where augmentation has no or little effect on the model's performance. Not all models are open to data augmentation. Graph neural networks (GNN), for instance, are invariant to representation shuffle. GNNs are thus incompatible with existing data augmentation methods used for natural language processing or images.

A third issue is that the training procedure 315 of a model defines an important aspect in modelling. Frequently, there are too many variables that may influence a model's decision. This may explain why hyper-parametrization optimization strategies may be required to improve models for performance or efficiency. Apart from the selected model, the question of the data split between train and test sets also plays a significant role. Several methods can be used from fully leave-one out, random-split to K-fold cross-validation to simulate and estimate the model quality on unseen data. In the end, a model's prediction is just an educated guess depending on the used training conditions, model size, optimization parameters, and data split. Upon completion, it is impossible to know if the best model was indeed trained. one assume, however, that the computed model is the best model for the used testing points. This is a general limitation of a data modeling approach as one should not necessarily expect that the performance results will be the same for all of parts of future unseen data. It should be noted that future performances may also vary considerably, depending on the evaluated sample size for the unseen data as well as a possible sample bias introduced in the unseen data. One way to partially solve these shortcomings is by predicting an accurate theoretical endpoint as standardized metric to evaluate a model. An example of such endpoint is the molecular weight of a molecule in chemistry.

In the field of chemical species and chemical reaction digital modeling using neural network, there are three main branches.

The first branch is learning models from graph neural networks (GNNs). To compute the atom properties any molecular input format can be used. This format cannot be readily augmented and is difficult for smaller datasets as frequently seen in all chemistry.

The second branch is NLP methods based upon line augmentation strings (such as the SMILES format), where the chemistry is exclusively learned from this syntax. This method has the benefit of data augmentation because the same molecule can be written by writing a new sentence in a different rule-based order (sentence grammar).

The third branch is image convolution neural networks learning and predicting from molecular images.

Such approaches require abundant datasets, which are rare in the fields of fragrance design and olfactometry, perfumery, fine fragrance perfumery and flavor design. Without abundant datasets, the use of neural network technologies can lead to inefficient models due to the risk of memorization by the network given the number of parameters to be considered.

Furthermore, the input of such graph neural networks is a conversion of a molecule into a specified input format, usually SMILES format of molecular structures, that are inadequate to create efficient chemical species features of chemical reaction prediction models.

Ensembling is a technique that consists in training several models (usually called base models or weak learners) and at inference time aggregating their outputs with some voting mechanism.

This technique is widely used by practitioners (notably to obtain winning solutions in many machine learning competitions), and it is often a key step to improve final performance.

Even though using ensembles is very popular, finding the best ensembling procedure to build, train and combine the base models is in general not trivial. Traditionally, ensembling techniques have been trying to produce a diverse (or complementary) collection of base models, and to combine them using some voting technique, usually meant to reduce the bias and/or the variance of the resulting system. A host of different techniques can be used to train diverse models. For example, bagging (with bootstrap resampling) introduces diversity via sampling of the training dataset and boosting introduces diversity by training models in sequence in a way such that each model has the incentive to compensate for the errors made by the preceding ones. Voting techniques can consist of simple averaging, majority voting (for classification), or stacking, whereby the final prediction is produced by a meta-model that is trained to combine the base models on some held-out dataset.

Although these techniques can work very well in practice, they mostly consist in hand-crafted heuristics made to enforce diversity or complementarity among the base models. Although these heuristics can be used to make base models complementary—for instance during training (e.g., in boosting) or at inference time (e.g., in stacking); the models are not directly learning how to best complement each other. In particular, the models are not able to explicitly capture the fact that they are part of an ensemble.

To address this challenge, proposals have been made to train all of the base models jointly (or end-to-end). In the context of neural networks, that means considering each of the base models as a part of a larger neural network and training them all jointly using a common loss.

Interestingly, this blurs the notions of ensembles and multi-head (or multi-branch) networks, as each of the base models can now be seen as a separate branch in a single neural network model. This end-to-end approach is attractive, but it is known that blindly optimizing for a global loss on the whole system often does not provide the best results, and it is usually better to perform some amount of individual training (often controlled by specific terms in the loss) of the base models.

Currently, the best-known ways to train such end-to-end models is often to try different interpolations between individual and global (or distillation) loss terms, and in general the best approaches appear to be problem and model dependent.

### SUMMARY OF THE INVENTION

The present invention aims at addressing all or part of these drawbacks.

According to a first aspect, the present invention aims at a method to predict at least one physico-chemical and/or odor property value for a chemical structure or composition, comprising the steps of:
defining, upon a computer interface, a digitized representation of a chemical structure or composition,
executing, by a computing device, upon the digitized representation defined, an end-to-end trained ensemble neural network or multi-branch neural network model to predict at least one physico-chemical and/or odor property value for a chemical structure or composition,
providing, upon a computer interface, the at least one physico-chemical and/or odor property value for a chemical structure or composition,
   further comprising the steps of:
providing a set of exemplar data, comprising at least one set of inputs, said inputs corresponding to digitized representations of chemical structures or compositions, and at least one set of outputs, said outputs corresponding to physico-chemical and/or odor properties associated with the set of inputs, to an end-to-end ensemble neural network or multi-branch neural network device comprising:
   several neural network sub-devices, each sub-device being configured to provide an independent prediction based upon the exemplar data,
   a layer configured to output at least one value based on, or representative of, the distribution of said independent predictions and
   said layer comprising a sampling device configured to output at least one random value as a function of a probability distribution representative of the distribution of independent predictions, said output random values being computed in a differentiable way and used for backpropagation within the end-to-end ensemble neural network or multi-branch neural network device,
operating the end-to-end ensemble neural network or multi-branch neural network device based upon the set of exemplar data and
obtaining the trained end-to-end ensemble neural network or multi-branch neural network model configured to predict physico-chemical and/or odor properties for input digitized representations of chemical structures or compositions.

Such provisions allow for the accurate prediction of physico-chemical and/or odor property values for defined chemical structures or compositions.

Such provisions allow, as well, for much greater prediction stability, reliability, as well as improved training speed and overall performance and the provision of a metric of variance representative for the model uncertainty. Such embodiments thus allow resource savings, in terms of computation time or power, as well as in terms of model complexity. Typically, current approaches require the use of numerous models and iterations to obtain a reliable prediction model.

Furthermore, such provisions allow the trained model to reach higher accuracies than competing approaches that either need to engineer diversity among the base models, or that rely on fine-tuned loss functions to balance the objectives of training the individual models along with the ensemble.

Such provisions also offer a simple means to regularize the ensemble by introducing noise. Finally, such provisions allow for more stable training dynamics and better individual base models. This approach does not require any extra tuning, and it does not introduce new learnable parameters.

In particular embodiments, at least one set of inputs of the exemplar data corresponds to hash vectors of at least one atomic property in a chemical structure or composition, the method further comprising, upstream of the step of executing, a step of converting the defined digitized chemical structure or composition into a set of hash vectors of at least one atomic property representative of the digitized chemical structure or composition, said set of hash vectors being used as input during the step of executing.

Such provisions prove particularly efficient in increasing the reliability of the results of prediction in the context of physico-chemical and/or odor properties prediction.

In particular embodiments, at least one hash vector of an atomic property is representative of one of the following:
atomic number of the corresponding atom,
atomic symbol of the corresponding atom,
mass of the atom,
explicit map number,
row index in the periodic system,
column index in the periodic system,
total number of hydrogens on the atom,
implicit number of hydrogens on the atom,
explicit number of hydrogens on the atom,
degree of the atom,
total degree of the atom,
the valence state of the atom,
the implicit valence of the atom,
the explicit valence of the atom,
formal charge on the atom,
partial charge on the atom,
electronegativity on the atom,
number of bonds by bond type,
number of neighbors by atomic number, wild card,
number of neighbors by bond type plus atomic number, wild card,
number of neighbors by wild card,
value to indicate aromaticity,
value to indicate aliphatic atom,
value to indicate a conjugated atom,
value to indicate cyclic atom,
value to indicate a macrocyclic atom,
value to indicate a geometrically constraint atom,
value to indicate electron withdrawing atom,
value to indicate an electron donating atom,
value to indicate the reaction site,
value to indicate hydrogen bond donor,
value to indicate hydrogen acceptor,
value to indicate the multivalence as hydrogen bond donor,
value to indicate the multivalence as hydrogen bond acceptor,
number of cycles on the atom,
ring size on the atom,
hybridization state of the atom,
values to indicate the atomic geometry,
number of electrons in the atomic orbitals,
number of electrons in lone pairs,
radical state,
the isotope on the atom,
atom center symmetric functions,
value for relative stereochemistry as chi clockwise, chi anticlockwise,
value for absolute stereochemistry,
value for absolute stereochemistry,
value for double bond stereochemistry,
value for priority for determination of stereochemistry,
value representative of a positive or negative impact of an atom upon a determined training target, to indicate knowledge-based enrichment contribution,
value representative of a positive or negative impact of an atom upon a determined training target, to indicate knowledge-based dilution contribution and/or
value for ring stereochemistry.

In particular embodiments, at least one hash vector of a bond property is representative of one of the following:
bond order,
bond type,
stereochemistry of the bond:
   bond direction for tetrahedral stereochemistry,
   bond direction for double bond stereochemistry or
   bond direction for spatial orientation,
atomic number(s) for the “from” and/or “to” atoms,
atomic symbols for the “from” and/or “to” atoms,
dipole moment in the bond,
quantum-chemical properties:
   electron density in the bond,
   electron configuration of the bond,
   bond orbitals,
   bond energies,
   attractive forces,
   repulsive forces,
bond distance,
aromatic bond,
aliphatic bond,
ring properties of the bond:
   number of rings on the bond,
   ring size(s) of the bond,
   smallest ring size of the bond,
   largest ring size of the bond,
rotatable bond,
spatially constrained bond,
hydrogen bonding properties,
ionic bonding properties,
bond order for reactions, including the “null” bond to identify a broken/formed bond in a reaction:
   bond order in reagents,
   bond order in intermediate products or
      bond order in transition states.

In particular embodiments, at least one output value representative of the distribution is representative of a dispersion of the distribution.

In particular embodiments, the end-to-end ensemble neural network or multi-branch neural network device is trained to minimize at least one value representative of the dispersion of the distribution.

In particular embodiments, at least one odor property is representative of:
an insect repellent capability value,
a sensory property value,
a biodegradability value,
an antibacterial value,
an odor detection threshold value,
an odor strength value,
a top-heart-base value,
a hazard value,
a biological activity for taste
a biological activity for olfaction,
a biological enhancing or modulating taste activity,
a biological enhancing or modulating olfaction activity and/or
an olfactive smell description.

In particular embodiments, at least one physical property is representative of:
a boiling point value,
a melting point value,
a water solubility value,
a Henry constant value,
a vapor pressure value,
a volatility value or
a headspace concentration value.

In particular embodiments, at least one neural network device is:
a recursive neural network device,
a graph neural network device,
a variational autoencoder neural network device or
an autoencoder neural network device.

In particular embodiments, the method object of the present invention comprises, upstream of the step of providing, a step of atom or bond relationship vector augmentation.

Such provisions allow for the use of initially considerably more limited datasets than what is currently required for neural network applications. Indeed, one molecular structure, represented by a one or an augmented series of hashes, can be augmented up to a maximum of times corresponding to the number of hashes of the series. Thus, one molecular structure can become several inputs in the natural language processing application.

In particular embodiments, the step of atom or bond relationship vector augmentation comprises a step of horizontal augmentation, configured to provide several vectors representing a single digitized representation of a molecular structure or composition, each vector representing a particular representation of the canonical representation molecular structure or composition, each vector being treated as a single input during the step of providing.

Such provisions allow for the use of initially considerably more limited datasets than what is currently required for neural network applications. Indeed, one molecular structure, represented by a one or an augmented series of hashes, can be augmented up to a maximum of times corresponding to the number of hashes of the series. Thus, one molecular structure can become several inputs in the natural language processing application.

In particular embodiments, the step of atom or bond relationship vector augmentation comprises a step of vertical augmentation, to create several groups of several horizontal augmentations, representing a unique molecular structure or composition, each group being treated as a single input during the step of providing.

Such provisions allow for the use of initially considerably more limited datasets than what is currently required for neural network applications. Indeed, one molecular structure, represented by a one or an augmented series of hashes, can be augmented up to a maximum of times corresponding to the number of hashes of the series. Thus, one molecular structure can become several inputs in the natural language processing application.

According to a second aspect, the present invention aims at a method to efficiently assemble chemical structures or compositions, comprising:
a step of executing a method object of the present invention and
a step of assembling a chemical structure or composition associated to an output obtained during the step of obtaining.

Such provisions allow for the materialization of the chemical structure for which an odor property prediction is performed.

According to a third aspect, the present invention aims at a system to predict at least one physico-chemical and/or odor property value for a chemical structure or composition, comprising the means of:
defining, upon a computer interface, a digitized representation of a chemical structure or composition,
executing, by a computing device, upon the digitized representation defined, an end-to-end trained ensemble neural network or multi-branch neural network model to predict at least one physico-chemical and/or odor property value for a chemical structure or composition,
providing, upon a computer interface, the at least one physico-chemical and/or odor property value for a chemical structure or composition,
   said system comprising the means of:
providing a set of exemplar data, comprising at least one set of inputs, said inputs corresponding to digitized representations of chemical structures or compositions, and at least one set of outputs, said outputs corresponding to physico-chemical and/or odor properties associated with the set of inputs, to an end-to-end ensemble neural network or multi-branch neural network device comprising:
   several neural network sub-devices, each sub-device being configured to provide an independent prediction based upon the exemplar data,
   a layer configured to output at least one value based on, or representative of, the distribution of said independent predictions and
   said layer comprising a sampling device configured to output at least one random value as a function of a probability distribution representative of the distribution of independent predictions, said output random values being computed in a differentiable way and used for backpropagation within the end-to-end ensemble neural network or multi-branch neural network device,
operating the end-to-end ensemble neural network or multi-branch neural network device based upon the set of exemplar data and
obtaining the trained end-to-end ensemble neural network or multi-branch neural network model configured to predict physico-chemical and/or odor properties for input digitized representations of chemical structures or compositions.

The advantages of the system object of the present invention are similar to the advantages of the method object of the present invention. Furthermore, all embodiments of the method object of the present invention may be reproduced, mutatis mutandis, in the system object of the present invention.

### BRIEF DESCRIPTION OF THE DRAWINGS

Other advantages, purposes and particular characteristics of the invention shall be apparent from the following non-exhaustive description of at least one particular embodiment or succession of steps of the present invention, in relation to the drawings annexed hereto, in which:

FIG. 1 shows, schematically, a first particular succession of steps of the method object of the present invention,

FIG. 2 shows, schematically, a particular embodiment of the system object of the present invention,

FIG. 3 shows, schematically, a general overview of machine learning systems,

FIG. 4 shows, schematically, a second particular succession of steps of the method object of the present invention,

FIG. 5 shows, schematically, a detailed view of a particular embodiment of new neural network layers used during the training of an end-to-end ensemble neural network or multi-branch neural network device,

FIG. 6 shows, schematically, a second particular succession of steps of the method object of the present invention,

FIG. 7 shows, schematically, a particular succession of steps to obtain a hash vector used by the system or method object of the present invention,

FIG. 8 shows, schematically, an example of implementation of the training method object of the present invention,

FIG. 9 to 11 show performance results on three odor properties,

FIGS. 12 to 14 show, schematically, training architectures that may be used to select, classify or predict odor properties and/or physico-chemical properties of chemical structures and

FIG. 15 shows, schematically, a particular training architecture that may be used to classify chemical structures.

### DETAILED DESCRIPTION OF THE INVENTION

This description is not exhaustive, as each feature of one embodiment may be combined with any other feature of any other embodiment in an advantageous manner.

Various inventive concepts may be embodied as one or more methods, of which an example can be provided. The acts performed as part of the method may be ordered in any suitable way.

Accordingly, embodiments may be constructed in which acts are performed in an order different than illustrated, which may include performing some acts simultaneously, even though shown as sequential acts in illustrative embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or lists of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should be noted at this point that the figures are not to scale.

As used herein, the term “ingredient” designates any ingredient, preferably presenting a flavoring or fragrance capacity. The terms “compound” or “ingredient” designate the same items as “volatile ingredient.” An ingredient may be formed of one or more chemical molecules.

The term composition designates a liquid, solid or gaseous assembly of at least two fragrance or flavor ingredients or one fragrance or flavor ingredient and a neutral solvent for dilution.

As used herein, a “flavor” refers to the olfactory perception resulting from the sum of odorant receptor(s) activation, enhancement, and inhibition (when present) by at least one volatile ingredient via orthonasal and retronasal olfaction as well as activation of the taste buds which contain taste receptor cells. Accordingly, by way of illustration and by no means intending to limit the scope of the present disclosure, a “flavor” results from the olfactory and taste bud perception arising from the sum of a first volatile ingredient that activates an odorant receptor or taste bud associated with a coconut tonality, a second volatile ingredient that activates an odorant receptor or taste bud associated with a celery tonality, and a third volatile ingredient that inhibits an odorant receptor or taste bud associated with a hay tonality.

As used herein, a “fragrance” refers to the olfactory perception resulting from the aggregation of odorant receptor(s) activation, enhancement, and inhibition (when present) by at least one volatile ingredient. Accordingly, by way of illustration and by no means intending to limit the scope of the present disclosure, a “fragrance” results from the olfactory perception arising from the aggregation of a first volatile ingredient that activates an odorant receptor associated with a coconut tonality, a second volatile ingredient that activates an odorant receptor associated with a celery tonality, and a third volatile ingredient that inhibits an odorant receptor associated with a hay tonality.

As used herein, an “odor property” or “olfactive property” refers to any psychophysical property of an ingredient or composition. Namely, such properties refer to how the human body reacts to the physical presence of an olfactory ingredient or composition, considering that such psychophysical properties are directly link to the ability of the ingredient or composition to easily penetrate and by in proximity contact to the olfactory receptors present in human body.

As used herein, the terms “means of inputting” is, for example, a keyboard, mouse and/or touchscreen adapted to interact with a computing system in such a way to collect user input. In variants, the means of inputting are logical in nature, such as a network port of a computing system configured to receive an input command transmitted electronically. Such an input means may be associated to a GUI (Graphic User Interface) shown to a user or an API (Application programming interface). In other variants, the means of inputting may be a sensor configured to measure a specified physical parameter relevant for the intended use case.

As used herein, the terms “computing system” or “computer system” designate any electronic calculation device, whether unitary or distributed, capable of receiving numerical inputs and providing numerical outputs by and to any sort of interface, digital and/or analog. Typically, a computing system designates either a computer executing a software having access to data storage or a client-server architecture wherein the data and/or calculation is performed at the server side while the client side acts as an interface.

As used herein, the terms “digital identifier” refers to any computerized identifier, such as one used in a computer database, representing a physical object, such as a flavoring ingredient. A digital identifier may refer to a label representative of the name, chemical structure, or internal reference of the flavoring ingredient.

In the present description, the term “materialized” is intended as existing outside of the digital environment of the present invention. “Materialized” may mean, for example, readily found in nature or synthesized in a laboratory or chemical plant. In any event, a materialized composition presents a tangible reality. The terms “to be compounded” or “compounding” refer to the act of materialization of a composition, whether via extraction and assembly of ingredients or via synthetization and assembly of ingredients.

As used herein, the terms “atomic properties” refer to the properties of atoms and/or bonds attached to any atoms regardless of their molecular use context. As such, atomic properties refer to an absolute description of features of atoms, as opposed to the relative description of atoms within a molecule in the broader context of the molecule such atoms are a part of.

As used herein, the terms “activation function” defines, in a neural network, how the weighted sum of the input is transformed into an output from a node or nodes in a layer of the network. These activation functions may be defined by layers in the network or by arithmetic solutions in the loss functions.

As used herein an “end-to-end ensemble neural network or multi-branch neural network device” refer to a group of independent neural network devices collaborating to provide outputs as well as a single neural network devices comprising independent branches collaborating to provide outputs.

As used herein, the terms “atomic properties” refer to the properties of atoms and/or bonds attached to any atoms regardless of their molecular use context. As such, atomic properties refer to an absolute description of features of atoms, as opposed to the relative description of atoms within a molecule in the broader context of the molecule such atoms are a part of.

The embodiments disclosed below are presented in a general manner.

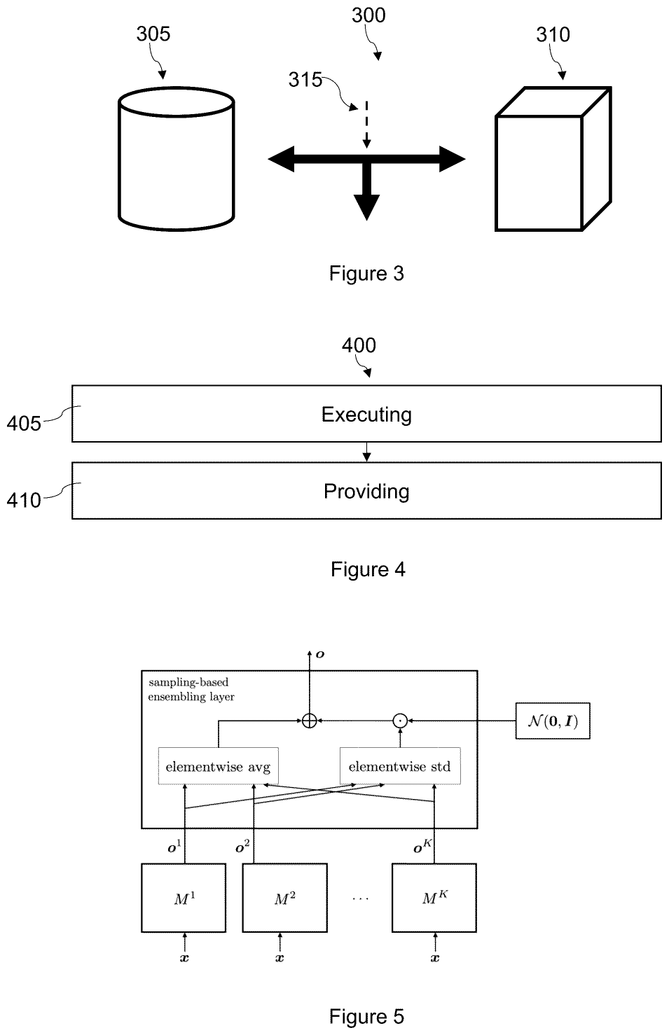

FIG. 3 shows a general view of machine learning key components.

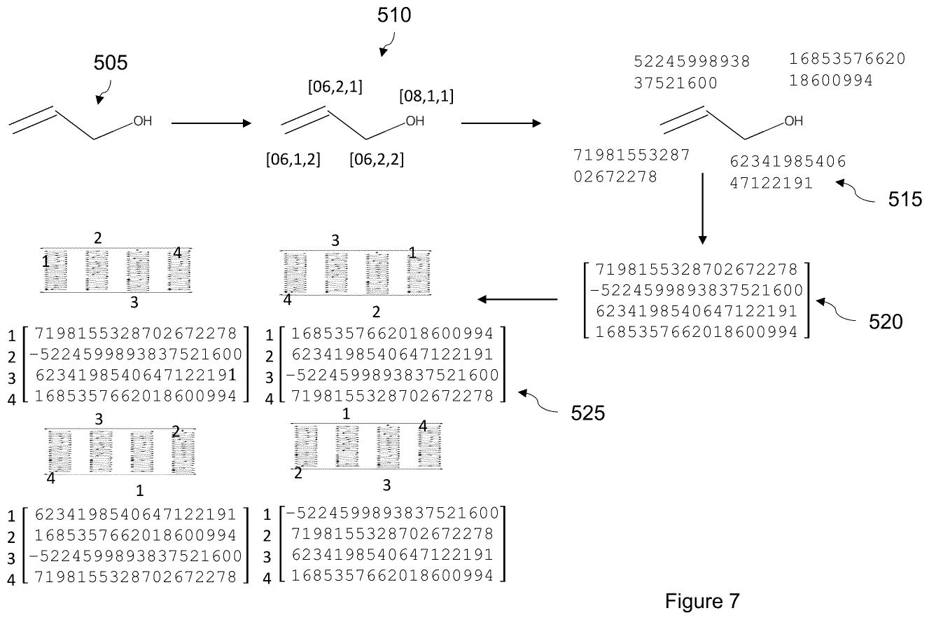

FIG. 5 shows, schematically, a particular embodiment of two layers of an end-to-end ensemble neural network or multi-branch neural network training device object of the present invention. FIG. 5 also helps in understanding the technical contribution of the present invention. The underlying theory of the model shown in FIG. 5 is presented below.

Let Ε be an ensemble (or multi-branch) neural network composed K base models M^{k}, k=1 . . . K. The present approach can be seen as a new neural network layer combining the vector outputs of several base models into one. At training time, it proceeds as follows:

Let x be one input if the neural network.

The k-th base model outputs o^{k}=M^{k}(x)∈R^{h}, where h is the output dimensionality of the base models. The layer takes in o^{k}, k=1 . . . K in input, and it outputs o˜D(g(o¹, . . . , o^{K})), where ˜ denotes differentiable sampling, D is a multivariate distribution, g is a function mapping the vectors o^{k} to the distribution's parameters, and o∈R^{h} has the same dimension as the individual input vectors. Using the output o of this layer, the final output of the network ŷ can for instance be obtained as ŷ=f(o) where f is a function providing the right output format for the task at hand (such as softmax for classification). At inference time, the layer outputs, for example, the mean of D(g(o¹, . . . , o^{K})) instead of random samples.

Using a reparameterization trick, sampling can be done in a differentiable way, so it is compatible with neural network training based on gradient descent. Therefore, contrary to traditional ensembling methods such as Bagging or Stacking that separate the training of each model in the ensemble, the present layer ensures that gradients are provided to all base models for all training samples, which results in a form of end-to-end training.

There are different options for the computation done by this layer, specified by D and g. As an example, a simple variant is shown on FIG. 5, where D is Gaussian distribution parameterized by a diagonal covariance matrix, and the function g(o¹, . . . , o^{K}) computes the parameters p E R^{h} and a E R^{h}, such that oi˜N(μᵢ, σ²ᵢ).

A few different ways of constructing and sampling from D(g(o¹, . . . , o^{K})) are disclosed below.
Mean: No sampling is done, the average of every base model is taken and used to compute an output. It boils down to training a multi-headed neural network where the outputs of the heads are simply averaged. More precisely,

$$o = \frac{1}{K} {\sum }_{k = 1}^{K} {o}^{k} .$$

Diagonal: This corresponds to the example shown in FIG. 5. During training, from the outputs o^{k} of the base models, the following values are computed:

$$μ = \frac{1}{K} {\sum }_{k = 1}^{K} {o}^{k}$$

and a such that

$${σ}_{i} = \sqrt{\frac{1}{K} {\sum }_{k = 1}^{K} {( {μ}_{i} - {σ}_{i}^{k} )}^{2}} .$$

A sample ϵ˜N(0, 1) is then produced and the layer's output is given by oᵢ=μᵢ+ϵᵢσᵢ, so o is distributed as N(μ, diag(σ²ᵢ)). At inference, the output of the layer is simply o=μ.
Diagonal parameterized: this approach similar to Diagonal, but it instead computes μ=l_{θ}(∥=ₖ₌₁^{K}o^{k}) and σᵢ=l_{y} (∥ₖ₌₁^{K}o^{k}) where l_{θ} and l_{γ} are two learnable functions (e.g., made fully connected layers) that output μ and σ directly. This way of specifying the distribution D(g(o¹, . . . , o^{K})) is reminiscent of a VAE with the difference lying in the fact that this layer computes p and a from several underlying base models.
Full covariance: This is an extension of Diagonal, where instead of using a diagonal covariance matrix, the sampling is done using a full covariance matrix. Here too, during training, the layer computes

$$μ = \frac{1}{K} {\sum }_{k = 1}^{K} {o}^{k} .$$

In principle, the covariance matrix would have to be computed as

$$\sum = \frac{1}{K} {\sum }_{k = 1}^{K} ( μ - {o}^{k} ) {( μ - {o}^{k} )}^{T} .$$

However, to apply the reparameterization trick in this setup, the layer needs to compute samples o=μ+Rϵ, where ϵ˜ N(0, 1) and R is commonly obtained from the Cholesky decomposition Σ=RR^{T}. This is problematic because the Cholesky decomposition requires E to be positive definite. A workaround is to compute the decomposition on Σ′=Σ+τI for some small τ∈R+. In practice, however, it can be observed this workaround to cause numerical issues affecting the results. Instead, a simpler approach may be used and bypass the computation of Σ and the Cholesky decomposition altogether. By noticing that

$$\sum = \frac{1}{K} {R}^{′} {R}^{′ T} ,$$

with R′ having (o¹, . . . , o^{K})) as columns, it can be computed

$$o = μ + \frac{1}{\sqrt{L}} {R}^{′} ϵ ,$$

with ϵ˜N(0, 1). Here too, at inference time the layer simply returns o=p.

The performance of this architecture can be evaluated the performance of our approach on the CIFAR-10 image classification task. Each competing model is trained using 5 random seeds and 120 epochs. The test loss is computed on the whole test set with the usual split for CIFAR-10, with train and test sets consisting of respectively 50 000 and 10 000 images.

The training method object of the present invention can be compared against different ensembling methods, in order to evaluate sampling as a new technique to train ensembles end-to-end. All ensembling methods use K=8 base models, which are standard CNNs containing ReLU and Batch normalization layers. Each base model has 68 906 parameters, and so each ensemble has a total of 551 248 parameters. Different variants, described above, which refers to the parameterized isotropic variant with a multilayer perceptron used for the function I(⋅) are evaluated. It is observed that when Diagonal Sampling is used the training can be unstable at the beginning, if a uniform based weight initialization is used. This is due to the initial base models not being diverse enough at the beginning of training, resulting in a close-to-zero standard deviation that makes the Gaussian sampling prone to numerical instabilities. Therefore, Gaussian or orthogonal based initializations can be used, which do not seem to suffer from this issue. In this particular embodiment, the version of Bagging which is based on random initialization of the network's weights, along with random shuffling of the data points is used. Finally, a Negative Correlation Learning (“NCL”) is used as in the equation below:

$$λ L ( {y}_{i} , {\hat{y}}_{i} ) + ( 1 - λ ) \frac{1}{K} {\sum }_{j = 1}^{K} L ( {y}_{i} , {\hat{y}}_{i}^{k} )$$

where L is a loss function, and K, y, ŷᵢ and ŷₖ denote respectively the number of base models in the ensemble, the i-th target, the i-th prediction of the ensemble, j is the index of the j-th sub-device, and the prediction of the k-th base model. The second term measures the diversity between the ensemble members, and λ is a hyper-parameter that needs to be tuned. Intuitively, λ=0 corresponds to individual training and λ=1 corresponds to end-to-end training.

In addition to ensembling methods, the present results can be compared with that obtained by a standalone CNN of similar capacity as the ensemble, both without (“Simple”) and with Dropout (“Simple+Dropout”). This CNN has a similar structure to the CNNs used for base models, but it has 506 290 parameters, which can be obtained by increasing the depth and the number of channels.

The validation accuracies of the different models can be used as a measure of performance. The coefficient of variation provides a measure of the diversity among the ensemble members throughout training. It is computed as the average of the elementwise standard deviation rescaled by the mean of o¹, . . . , o^{K}. Finally, the average test accuracy of the base models can be used as a metric of performance. This measures the distillation during training, i.e., how performant each independent base model is on the test set.

From this comparison, it can be seen that:
Mean performs better than Single which indicates that multiple heads can already provide some benefits even when the outputs of the base models are simply averaged during training. However, Mean is deterministic and does not inject any noise. By comparing Diagonal and Mean one can see the benefits brought by sampling during training compared to simple averaging, as Diagonal clearly provides better results.
Full Covariance Sampling ends up having a better validation accuracy than the simpler Diagonal alternative. The coefficient of variation is bigger in the case of the Full Covariance Sampling compared to the Diagonal, which seems to indicate that Full Covariance benefits from a better diversity. Additionally, comparing the two sampling methods, Full Covariance Sampling has a better aggregated test accuracy from approximately the 30th epoch of training.

However, it can be noted that Full Covariance only gets better than Diagonal in terms of test accuracy after around the 60th epoch. In other words, even when it has worse test accuracy, Full Covariance has better averaged individual test accuracy than Diagonal. Therefore, Full Covariance offers better distillation properties. Overall, it appears here that sampling from a richer distribution gives better results. However, Full Covariance only outperforms the other methods after a few tens of epochs, and it is more is more computationally costly. Overall, the gains come at the expense of more computations for the same number of parameters.
By comparing Diagonal MLP and Diagonal, it one can note a net advantage for the latter, which suggests that using such an MLP-parameterized function to compute the parameters D from a concatenated vector of o¹, . . . , o^{K} is worse than directly computing the elementwise mean and standard deviation of the vectors. It is however not clear if the performance gap comes from the inductive bias of the direct (non-parametric) option or from some other factor. Hence, one suggest directly computing the parameters of D, which also has the merit of not adding new learnable parameters.
It is presumed that the present training device first works as a regularization mechanism, which allows end-to-end training of ensembles that would otherwise be prone to overfitting. Indeed, even if Mean already provides a net increase over Single, Mean underperforms Bagging in terms of test accuracy and distillation. Nonetheless, as soon as sampling is used, better test accuracy is obtained and distillation in favor of the sampling methods. Additionally, injecting noise in this way means that the sampling procedure adapts to the magnitude of o₁, . . . , o_{K} during training. In comparison, relying on a noise schedule would add some complexity and would require careful tuning.
The performance gap between Diagonal and Full Covariance indicates that the more sophisticated distribution gives better expressive power to the ensemble, which hints at the fact that the sampling distribution could capture parts of the data and would thus be doing more than being only a noise mechanism that provides regularization with a variable magnitude during training.
Single with Dropout is more regularized than Full Covariance (the other approaches overfit to the training set). However, Full Covariance still performs best on the test set. Therefore, it seems that our sampling-based approach can navigate different regions of the bias-variance trade-off.
NCL and Bagging perform similarly in terms of test accuracy, but NCL has a slightly better distillation and more diversity which illustrates the benefits of end-to-end ensembling. Nevertheless, on top of these advantages, the present method only requires choosing the right distribution as opposed to tuning a new hyper-parameter (as in the case of NCL).
Training a standalone CNN overfits, which can be alleviated with Dropout. Nevertheless, Single+Dropout is more unstable than the ones of Diagonal and Full Covariance which can be an issue when stability is important (as is often the case for deployment in industrial applications).

Finally, the table below, it can be seen that Full Covariance provides an advantage in terms of test accuracy over the competing methods.

| Model | Validation accuracy |
|---|---|
| Bagging | 81.6% ± 0.2 |
| Full covariance (present method) | 83.1% ± 0.3 |
| Diagonal MLP (present method) | 76% ± 0.5 |
| Diagonal (present method) | 82.4% ± 0.2 |
| Mean | 79.2% ± 0.3 |
| NCL | 81.7% ± 0.4 |
| Single | 77% ± 0.5 |
| Single + Dropout | 82.8% ± 1.1 |

The present approach is thus particularly useful for combining multiple branches of a neural network, which can be seen as a way to perform end-to-end training of an ensemble of neural networks. It consists of a new neural network layer, which takes as inputs several individual predictions coming from distinct base models (or branches) and uses differentiable sampling to produce a single output while offering regularization and distributing the gradient to all base models. This approach has multiple benefits.

First, it reaches higher accuracies than competing approaches that either need to engineer diversity among the base models, or that rely on fine-tuned loss functions to balance the objectives of training the individual models along with the ensemble.

Second, it offers a simple means to regularize the ensemble by introducing noise.

Third, it results in more stable training dynamics and better individual base models. This approach does not require any extra tuning, and it does not introduce new learnable parameters.

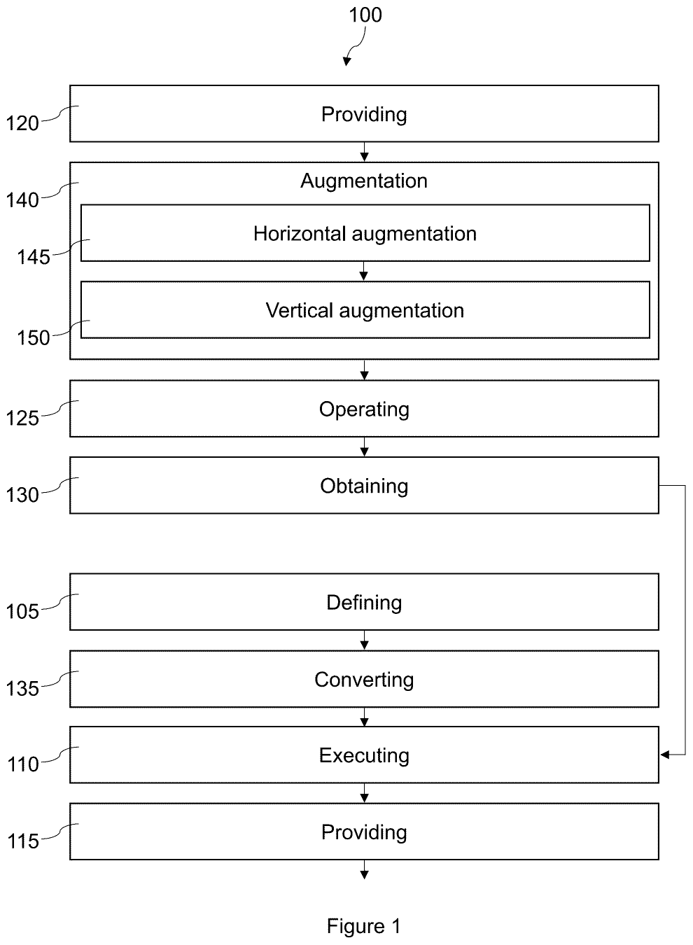

FIG. 1 shows a particular succession of steps of the method 100 object of the present invention. This method 100 to predict at least one physico-chemical and/or odor property value for a chemical structure or composition, comprises the steps of:
defining 105, upon a computer interface, a digitized representation of a chemical structure or composition,
executing 110, by a computing device, upon the digitized representation defined, an end-to-end trained ensemble neural network or multi-branch neural network model to predict at least one physico-chemical and/or odor property value for a chemical structure or composition,
providing 115, upon a computer interface, the at least one physico-chemical and/or odor property value for a chemical structure or composition,
   the method 100 object of the present invention further comprising the steps of:
providing 120 a set of exemplar data, comprising at least one set of inputs, said inputs corresponding to digitized representations of chemical structures or compositions, and at least one set of outputs, said outputs corresponding to physico-chemical and/or odor properties associated with the set of inputs, to an end-to-end ensemble neural network or multi-branch neural network device comprising:
   several neural network sub-devices, each sub-device being configured to provide an independent prediction based upon the exemplar data,
   a layer configured to output at least one value based on, or representative of, the distribution of said independent predictions and
   said layer comprising a sampling device configured to output at least one random value as a function of a probability distribution representative of the distribution of independent predictions, said output random values being computed in a differentiable way and used for backpropagation within the end-to-end ensemble neural network or multi-branch neural network device,
operating 125 the end-to-end ensemble neural network or multi-branch neural network device based upon the set of exemplar data and
obtaining 130 the trained end-to-end ensemble neural network or multi-branch neural network model configured to predict physico-chemical and/or odor properties for input digitized representations of chemical structures or compositions.

It should be noted that the layer configured to output at least one value based on, or representative of, the distribution of said independent predictions can either be understood as a layer providing a value representative of a distribution to be used by the sampling device or as a layer providing a value obtained from the sampling device.

By a “differentiable way”, it is meant a way to draw the samples from the distribution that makes it possible to compute the gradients of the layer output(s) with respect to the distribution's parameters. It also implies that these parameters are computed using differentiable functions of the outputs of the neural network sub-devices. This allows to obtain a “proper” neural network layer for which one can compute the gradient of the output(s) with respect to its input(s), which makes it possible to embed it in any larger neural network trained using backpropagation.

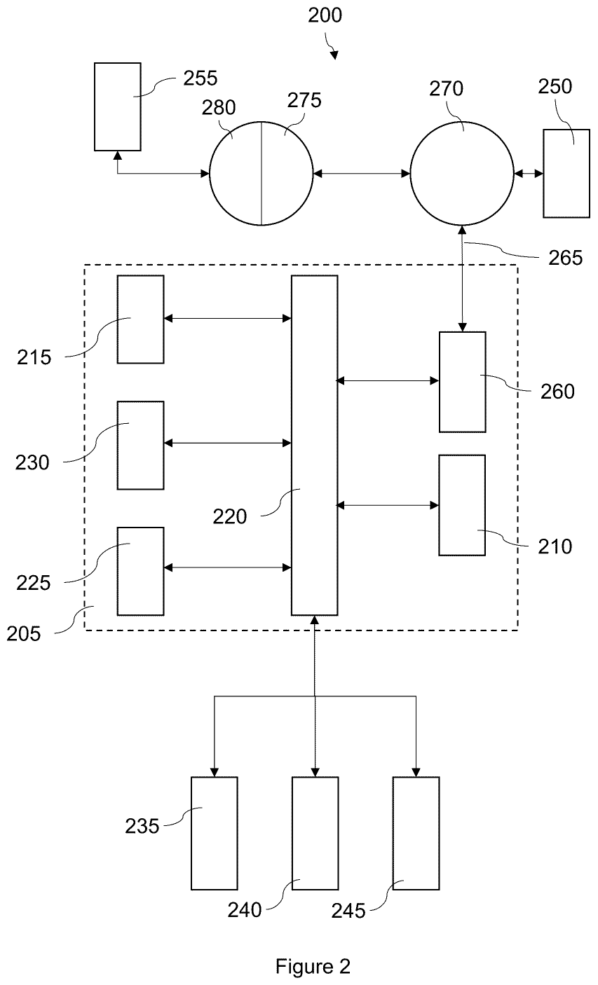

The step of defining 105 is performed, for example, by using an input device 240 coupled to I/O subsystem 220 such as disclosed in regard to FIG. 2.

During this step of defining 105, a chemical structure or a composition is defined.

A chemical structure is defined as molecular geometry and, optionally, the electronic structure of a target molecule. Molecular geometry refers to the spatial arrangement of atoms in a molecule and the chemical bonds that hold the atoms together and can be represented using structural formulae and by molecular models; complete electronic structure descriptions include specifying the occupation of a molecule's molecular orbitals. Structure determination can be applied to a range of targets from very simple molecules (e.g., diatomic oxygen or nitrogen), to very complex ones (e.g., such as protein or DNA).

A composition is defined as a sum of molecules or compounds, typically called flavor or fragrance ingredients.

During this step of defining 105, for example, a user may connect to a GUI and select existing chemical structures or design chemical structures by specifying the composing atoms and associated geometry. A user may alternatively connect to a GUI and select existing fragrance or flavor ingredients, each ingredient being associated with at least one chemical structure. Such selection or definition of chemical structures or compositions is performed with digital representations of the material equivalent of said chemical structures or compositions. Said representations may be shown as text and related to entries in computer databases storing, for each representation, a number of parameters.

The step of executing 110 is performed, for example, by one or more hardware processors 210, such as shown in FIG. 2, configured to execute a set of instructions representative of the trained end-to-end ensemble neural network or multi-branch neural network model. Particular embodiments for implementation of the step of executing 110 are disclosed above, in relation to FIG. 5 notably.

The input of the step of executing 110 is dependent on the parameters upon which the end-to-end ensemble neural network or multi-branch neural network device is operated to obtain an end-to-end ensemble neural network or multi-branch neural network model. For example, such parameters may correspond to:
a list of atoms constitutive a chemical structure, such as a molecule,
a list of atomic properties in a chemical structure,
a list of ingredients in a composition,
a list of molecules in a composition and/or
a list of molecules corresponding to ingredients in a composition.

The end-to-end ensemble neural network or multi-branch neural network model is configured to provide an output for a standardized input format. This standardized input format may correspond to digital representations of said atoms, atomic properties, molecules, ingredients, compositions and/or chemical structures. Such digital representations may correspond to character strings. Such strings may be concatenated to form unitary inputs representative of larger scale material items, such as several atoms forming a molecule, for example.

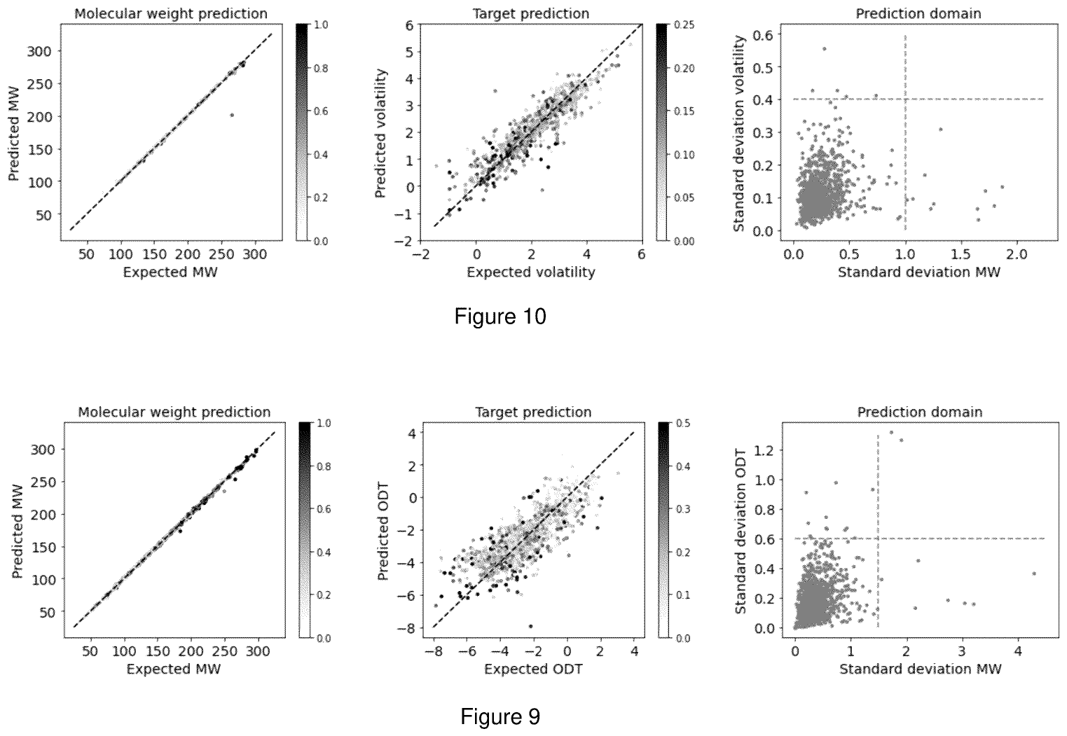

Examples of such inputs are shown in regard to FIGS. 7 and 12 to 14.

The step of providing 115 is performed, for example, by using an output device 235 coupled to I/O subsystem 220 such as disclosed in regard to FIG. 2.

In particular embodiments, this step of providing 115 shows, upon a GUI, the result of the prediction of the model based upon the defined chemical structure or composition fed to the model.

The step 120 of providing may be performed, via a computer interface, such as an API or any other digital input means. This step 120 of providing may be initiated manually or automatically. The set of exemplar data may be assembled manually, upon a computer interface, or automatically, by a computing system, from a larger set of exemplar data.

The exemplar data may comprise, for example:
at least one at digitized representation of a chemical structure or composition and
a value representative of an odor property or physico-chemical property associated with the chemical structure or composition.

Such an odor property may be, for example, a tonality of the chemical structure, an odor detection threshold value for the chemical structure, an odor strength (such as a classification of olfactive power into four classes of range intensities: odorless, weak, medium and strong classes of an ingredient or composition) for the chemical structure and/or a top-heart-base (such as the classification of the three range of long lastingness during evaporation of the ingredient or composition: top, heart, base classes of an ingredient or composition, in which “top” represents ingredients or compositions that can be smelled or determined by gas chromatography analysis until 15 min of evaporation, “heart” between 15 min to 2 hours and “base” more than 2 hours) value for the chemical structure. This list is not limitative, and any odor property known to the fields of fragrance and flavor design and associated industry may be associated with the hash vector.

An odor property may correspond to:
an insect repellent capability value,
a sensory property value,
a biodegradability value,
an antibacterial value,
an odor detection threshold value,
an odor strength value,
a top-heart-base value,
a hazard value,
a biological activity for taste
a biological activity for olfaction,
a biological enhancing or modulating taste activity,
a biological enhancing or modulating olfaction activity and/or
an olfactive smell description.

A physico-chemical may correspond to:
a boiling point value,
a melting point value,
a water solubility value,
a Henry constant value,
a vapor pressure value,
a volatility value or
a headspace concentration value.

The step 125 of operating may be performed, for example, by a computer program executed upon a computing system. During this step 125 of operating, the end-to-end ensemble neural network or multi-branch neural network device is configured to train based upon the input data. During this step 125 of operating, each neural network sub-device of the end-to-end ensemble neural network or multi-branch neural network device configures coefficients of the layers of artificial neurons to provide an output, these outputs forming a distribution of outputs. The values of statistical parameters representative of the distribution may be obtained and used in activation functions to be minimized.

Each neural network sub-device within the ensemble may be of the same type or different types.

In particular embodiments, at least one neural network sub-device is:
a recursive neural network device,
a graph neural network device,
a variational autoencoder neural network device or
an autoencoder neural network device.

In particular embodiments, at least two of the activation functions are representative of:
a means of the statistical distribution of the plurality of independent predictions,
the variance of the statistical distribution of the plurality of independent predictions and
optionally extended with additional activation functions, representative of:
   the skew of the statistical distribution of the plurality of independent predictions and/or
   the kurtosis of the statistical distribution of the plurality of independent predictions.

In particular embodiments, at least one output value representative of the distribution is representative of a dispersion of the distribution.

Such a value may correspond to, for example, the standard deviation of the outputs of the neural network sub-devices.

In particular embodiments, the end-to-end ensemble neural network or multi-branch neural network device is trained to minimize at least one value representative of the dispersion of the distribution.

The step 130 of obtaining may be performed, via a computer interface, such as an API or any other digital output system. The obtained trained model may be stored in a data storage, such as a hard-drive or database for example.

In particular embodiments, the neural network device obtained during the step 130 of obtaining is configured to provide, additionally, at least one value representative of the statistical dispersion of the output.

In particular embodiments, at least one set of inputs of the exemplar data corresponds to hash vectors of at least one atomic property in a chemical structure or composition, the method further comprising, upstream of the step 110 of executing, a step 135 of converting the defined digitized chemical structure or composition into a set of hash vectors of at least one atomic property representative of the digitized chemical structure or composition, said set of hash vectors being used as input during the step of executing.

A hash corresponds to the result of a hash function, which corresponds to any function that can be used to map data of arbitrary size to fixed-size values. Many such functions are known by persons skilled in the art, such as SHA-3, Skein or Snefru.

Such hash values can be organized into vectors that may be used by the end-to-end ensemble neural network or multi-branch neural network device.

To obtain such a hash vector representative of an atomic property in a chemical structure, a method comprising the following steps may be implemented:
a step of receiving, by a computing system, a digitized representation of a chemical structure, comprising at least one atomic property digital identifier and at least one atomic property digital identifier for said at least one atomic property digital identifier,
a step of determining, by a computing system, at least one value corresponding for at least one atomic or bond property of at least one atom or bond of the digitized representation of a chemical structure,
a step of hashing, by a computing system, of at least one determined value to form a unique character string fingerprinting at least one atomic property digital identifier and at least one associated one atomic property and
a step of providing, by a computing system, at least one hash to an ensemble of neural network devices to be trained.

The step of receiving is performed, for example, by any input device 240 fitting the particular use case. For example, during this step of receiving, at least one digitized representation of a chemical, atom or bond structure is input into a computer interface. Such an input may be entirely logical, such as by using an API (Application Programing Interface) or by interfacing said computing system to another computing system via a computer network. Such an input may also rely on a human-machine interface, such as a keyboard, mouse or touchscreen for example. The mechanism used for the step of receiving is unimportant with regards to the scope of the present invention.

Ultimately, the digitized representation of a chemical structure comprises essentially two types of data:
atom identifiers, corresponding to atoms that are part of the molecular structure, typically represented by at least one letter (such as “C” for carbon or “H” for hydrogen for example) and
at least one relationship for at least one atom identifier, defining if and how at least one atom is linked to other atoms of the molecule.

This digitized representation can take many forms, depending on the system. For example, the SMILES (for “Simplified Molecular Input Line Entry System”) format is a line notation of a molecular structure providing said two types of data. Another example is a molecular graph representation of the molecule. Another representation is the SDF (for “Structure Data File”) format defining the atoms with properties and the bond tables. Another representation is a full molecular matrix composed of the atomic numbers and the adjacency matrix defining the bonds.

Typically, the main digitized representation used in chemical reaction modeling and feature prediction is the SMILES format.

The step of determining is performed, for example, by one or more hardware processors 210, such as shown in FIG. 2, configured to execute a set of instructions representative of a computer software. During this step of determining, at least one atomic property can be read from the digitized representation of the chemical structure or determined via the execution of a dedicated algorithm or obtained from a dedicated third-party software.

The step of hashing is performed, for example, by one or more hardware processors 210, such as shown in FIG. 2, configured to execute a set of instructions representative of a computer software.

The output of the step of hashing is a given number of hashes, each hash being representative of one atom identifier as well as at least one associated atomic property of said identified atom. A chemical structure comprising several atoms is thus represented by a sentence of several hashes. Each hash acts as a unique fingerprint which is particularly useful for neural network applications. This means that, within a dataset, each atom can be represented by the corresponding hash key (the unique fingerprint).

Such hashes force sparsity in the network in an intrinsic manner.

During this step of hashing, a hash can be composed of the repeated values for the properties to define the property value in reagents, intermediates, transition states and products.

In particular embodiments, at least one of the atom properties hashed is representative of one of the following:
atomic number of the corresponding atom,
atomic symbol of the corresponding atom,
mass of the atom,
explicit map number,
row index in the periodic system,
column index in the periodic system,
total number of hydrogens on the atom,
implicit number of hydrogens on the atom,
explicit number of hydrogens on the atom,
degree of the atom,
total degree of the atom,
the valence state of the atom,
the implicit valence of the atom,
the explicit valence of the atom,
formal charge on the atom,
partial charge on the atom,
electronegativity on the atom,
number of bonds by bond type,
number of neighbors by atomic number, wild card,
number of neighbors by bond type plus atomic number, wild card,
number of neighbors by wild card,
value to indicate aromaticity,
value to indicate aliphatic atom,
value to indicate a conjugated atom,
value to indicate cyclic atom,
value to indicate a macrocyclic atom,
value to indicate a geometrically constraint atom,
value to indicate electron withdrawing atom,
value to indicate an electron donating atom,
value to indicate the reaction site,
value to indicate hydrogen bond donor,
value to indicate hydrogen acceptor,
value to indicate the multivalence as hydrogen bond donor,
value to indicate the multivalence as hydrogen bond acceptor,
number of cycles on the atom,
ring size on the atom,
hybridization state of the atom,
values to indicate the atomic geometry,
number of electrons in the atomic orbitals,
number of electrons in lone pairs,
radical state,
the isotope on the atom,
atom center symmetric functions,
value for relative stereochemistry as chi clockwise, chi anticlockwise,
value for absolute stereochemistry,
value for absolute stereochemistry,
value for double bond stereochemistry,
value for priority for determination of stereochemistry,
value representative of a positive or negative impact of an atom upon a determined training target, to indicate knowledge-based enrichment contribution,
value representative of a positive or negative impact of an atom upon a determined training target, to indicate knowledge-based dilution contribution and/or value for ring stereochemistry.

Regarding values representative of a positive or negative impact of an atom upon a determined training target, such values may be initialized by a user or trained as part of an auxiliary training method. Such values may be used at the atomic level or the molecular level.

An alternative approach to hashing comprises a step of assigning characters to each atomic property and a step of concatenation of said characters into a “word”. Such characters may correspond to characters within a SMILES string in which all characters not identified as chemical atomic characters are removed.

In particular embodiments, at least one of the bond properties hashed is representative of one of the following:
bond order,
bond type,
stereochemistry of the bond:
   bond direction for tetrahedral stereochemistry,
   bond direction for double bond stereochemistry or
   bond direction for spatial orientation,
atomic number(s) for the “from” and/or “to” atoms,
atomic symbols for the “from” and/or “to” atoms,
dipole moment in the bond,
quantum-chemical properties:
   electron density in the bond,
   electron configuration of the bond,
   bond orbitals,
   bond energies,
   attractive forces,
   repulsive forces,
bond distance,
aromatic bond,
aliphatic bond,
ring properties of the bond:
   number of rings on the bond,
   ring size(s) of the bond,
   smallest ring size of the bond,
   largest ring size of the bond,
rotatable bond,
spatially constrained bond,
hydrogen bonding properties,
ionic bonding properties,
bond order for reactions, including the “null” bond to identify a broken/formed bond in a reaction:
   bond order in reagents,
   bond order in intermediate products or
   bond order in transition states.

The step of obtaining is performed, for example, by using any output device 235 associated with an I/O subsystem 220, such as shown in FIG. 2.

In particular embodiments, the method to obtain a hash vector may further comprises:
a step of constructing, by a computing system, a chemical structure string fingerprint by association, in a single string, of at least two hashes corresponding to at least two atomic properties and
at least one step of augmentation, by a computing system, of at least one chemical structure string fingerprint, said augmented chemical structure string fingerprint being used during the step of providing.

The step of constructing is performed, for example, by one or more hardware processors 210, such as shown in FIG. 2, configured to execute a set of instructions representative of a computer software. During this step of constructing at least two hashes corresponding to at least two atom identifiers and associated features are associated, typically by concatenation of the respective hashes. The order of concatenation may follow concatenation rules that prevent neural network misinterpretation.

FIG. 7 represents, schematically, a particular embodiment of this method to obtain a hash vector representative of a chemical structure. As such, FIG. 7 represents:
a step 505 receiving an input chemical structure,
a step 510 of determining atom and/or bond properties in the received chemical structure, thus annotating a vector of input properties; the properties used here are [atomic number, degree, number of hydrogens],
a step 515 of property vector hashing to a single hashed key; alternately, a hash key identifying the atom type can be formed by simply concatenating the values, e.g. [06,1,1] becoming “0611”,
a step 520 of constructing a vector of the hashed keys for the atoms as specified by the atom order and
a step 525 of data augmentation on the vector can be applied by changing the atom order; the order may include the vector for the canonical order of atoms.

In particular embodiments, the method 100 object of the present invention comprises, upstream of the step 120 of providing input data to and end-to-end ensemble neural network or multi-branch neural network device, a step 140 of atom or bond relationship vector augmentation.

At least one step of augmentation 140 is performed, for example, by a computer software ran on a computing device, such as a microprocessor for example. During this step of augmentation 140, the order of the hashes of the constitutive hashes for a given molecular structure is shifted by one or more in the ordering of said constitutive hashes. That is to say, for example, that the last hash becomes the penultimate, the penultimate becomes the ante-penultimate and the first becomes the last or the other way around depending on the intended order of augmentation.

Such augmentations allow for the increase in sample size from the same chemical structure, which greatly improves the quality of the output of a neural network device.

In particular embodiments, the step 140 of atom or bond relationship vector augmentation comprises a step 145 of horizontal augmentation, configured to provide several vectors representing a single digitized representation of a molecular structure or chemical reaction, each vector representing a particular representation of the canonical representation molecular structure or chemical reaction, each vector being treated as a single input during the step of providing.

In particular embodiments, the step 140 of atom or bond relationship vector augmentation comprises a step 150 of vertical augmentation, create several groups of several horizontal augmentations, representing a unique molecular structure or chemical reaction, each group being treated as a single input during the step of providing.

Such a step 150 of vertical augmentation may be performed, for example, by a computer software executed by a computing system. This step 150 of vertical augmentation may be performed by grouping horizontal augmentations in single inputs, typically by concatenation of the hash keys representative of the atom and/or bond properties of a chemical structure. Such single inputs may be identical or different, by changing the order of concatenation for example.

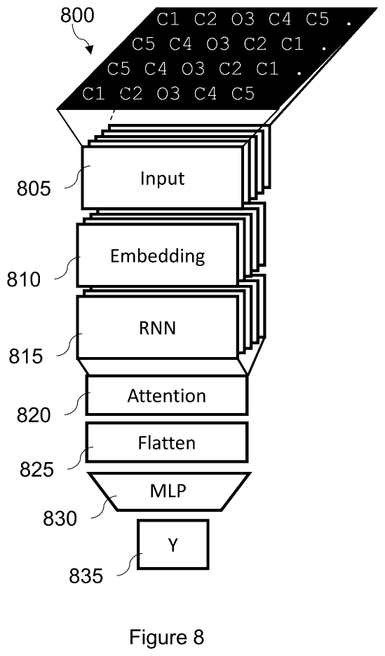

FIG. 6 shows another representation of a particular embodiment of the method 600 object of the present invention.

FIG. 6, in particular, shows the training of the model, comprising the steps of providing 120, operating 125 and obtaining 130.

During the step of providing 120, digitized representations 605 of chemical structures and known odor property values or physico-chemical property values 610 are used as input.

During the step of operating 125, an end-to-end ensemble neural network or multi-branch neural network device 615 is trained to output two values, 620 and 625, representative of the distribution of the individual outputs of neural network sub-devices constitutive of the end-to-end ensemble neural network or multi-branch neural network device 615, such as the mean and the standard deviation.

FIG. 2 represents, schematically, a particular embodiment of the system 200 object of the present invention. This system 200 to predict at least one physico-chemical and/or odor property value for a chemical structure or composition, comprising the means 205 of:
defining, upon a computer interface, a digitized representation of a chemical structure or composition,
executing, by a computing device, upon the digitized representation defined, an end-to-end trained ensemble neural network or multi-branch neural network model to predict at least one physico-chemical and/or odor property value for a chemical structure or composition,
providing, upon a computer interface, the at least one physico-chemical and/or odor property value for a chemical structure or composition,
   further comprises the means 205 of:
providing a set of exemplar data, comprising at least one set of inputs, said inputs corresponding to digitized representations of chemical structures or compositions, and at least one set of outputs, said outputs corresponding to physico-chemical and/or odor properties associated with the set of inputs, to an end-to-end ensemble neural network or multi-branch neural network device comprising:
   several neural network sub-devices, each sub-device being configured to provide an independent prediction based upon the exemplar data,
   a layer configured to output at least one value based on, or representative of, the distribution of said independent predictions and
   said layer comprising a sampling device configured to output at least one random value as a function of a probability distribution representative of the distribution of independent predictions, said output random values being computed in a differentiable way and used for backpropagation within the end-to-end ensemble neural network or multi-branch neural network device,
operating the end-to-end ensemble neural network or multi-branch neural network device based upon the set of exemplar data and
obtaining the trained ensemble neural network or multi-branch neural network device configured to predict physico-chemical and/or odor properties for input digitized representations of chemical structures or compositions.

FIG. 2 represents a block diagram that illustrates an example computer system with which an embodiment may be implemented. In the example of FIG. 2, a computer system 205 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

The computer system 205 includes an input/output (IO) subsystem 220 which may include a bus and/or other communication mechanism(s) for communicating information and/or instructions between the components of the computer system 205 over electronic signal paths. The I/O subsystem 220 may include an I/O controller, a memory controller and at least one I/O port. The electronic signal paths are represented schematically in the drawings, for example as lines, unidirectional arrows, or bidirectional arrows.

At least one hardware processor 210 is coupled to the I/O subsystem 220 for processing information and instructions. Hardware processor 210 may include, for example, a general-purpose microprocessor or microcontroller and/or a special-purpose microprocessor such as an embedded system or a graphics processing unit (GPU) or a digital signal processor or ARM processor. Processor 210 may comprise an integrated arithmetic logic unit (ALU) or may be coupled to a separate ALU.

Computer system 205 includes one or more units of memory 225, such as a main memory, which is coupled to I/O subsystem 220 for electronically digitally storing data and instructions to be executed by processor 210. Memory 225 may include volatile memory such as various forms of random-access memory (RAM) or other dynamic storage device. Memory 225 also may be used for storing temporary variables or other intermediate information during execution of instructions to be executed by processor 210. Such instructions, when stored in non-transitory computer-readable storage media accessible to processor 210, can render computer system 205 into a special-purpose machine that is customized to perform the operations specified in the instructions.

Computer system 205 further includes non-volatile memory such as read only memory (ROM) 230 or other static storage device coupled to the I/O subsystem 220 for storing information and instructions for processor 210. The ROM 230 may include various forms of programmable ROM (PROM) such as erasable PROM (EPROM) or electrically erasable PROM (EEPROM). A unit of persistent storage 215 may include various forms of non-volatile RAM (NVRAM), such as FLASH memory, or solid-state storage, magnetic disk, or optical disk such as CD-ROM or DVD-ROM and may be coupled to I/O subsystem 220 for storing information and instructions. Storage 215 is an example of a non-transitory computer-readable medium that may be used to store instructions and data which when executed by the processor 210 cause performing computer-implemented methods to execute the techniques herein.

The instructions in memory 225, ROM 230 or storage 215 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 205 may be coupled via I/O subsystem 220 to at least one output device 235. In one embodiment, output device 235 is a digital computer display. Examples of a display that may be used in various embodiments include a touch screen display or a light-emitting diode (LED) display or a liquid crystal display (LCD) or an e-paper display. Computer system 205 may include other type(s) of output devices 235, alternatively or in addition to a display device. Examples of other output devices 235 include printers, ticket printers, plotters, projectors, sound cards or video cards, speakers, buzzers or piezoelectric devices or other audible devices, lamps or LED or LCD indicators, haptic devices, actuators, or servos.

At least one input device 240 is coupled to I/O subsystem 220 for communicating signals, data, command selections or gestures to processor 210. Examples of input devices 240 include touch screens, microphones, still and video digital cameras, alphanumeric and other keys, keypads, keyboards, graphics tablets, image scanners, joysticks, clocks, switches, buttons, dials, slides.

Another type of input device is a control device 245, which may perform cursor control or other automated control functions such as navigation in a graphical interface on a display screen, alternatively or in addition to input functions. Control device 245 may be a touchpad, a mouse, a trackball, or cursor direction keys for communicating direction information and command selections to processor 210 and for controlling cursor movement on display 235. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 240 may include a combination of multiple different input devices, such as a video camera and a depth sensor.

In another embodiment, computer system 205 may comprise an internet of things (loT) device in which one or more of the output device 235, input device 240, and control device 245 are omitted. Or, in such an embodiment, the input device 240 may comprise one or more cameras, motion detectors, thermometers, microphones, seismic detectors, other sensors or detectors, measurement devices or encoders and the output device 235 may comprise a special-purpose display such as a single-line LED or LCD display, one or more indicators, a display panel, a meter, a valve, a solenoid, an actuator or a servo.

Computer system 205 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 205 in response to processor 210 executing at least one sequence of at least one instruction contained in main memory 225. Such instructions may be read into main memory 225 from another storage medium, such as storage 215. Execution of the sequences of instructions contained in main memory 225 causes processor 210 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage 215. Volatile media includes dynamic memory, such as memory 225. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 220. Transmission media can also take the form of acoustic or light waves, such as those generated during radio-wave and infra-red data communications.

Various forms of media may be involved in carrying at least one sequence of at least one instruction to processor 210 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 205 can receive the data on the communication link and convert the data to a format that can be read by computer system 205. For instance, a receiver such as a radio frequency antenna or an infrared detector can receive the data carried in a wireless or optical signal and appropriate circuitry can provide the data to I/O subsystem 220 such as place the data on a bus. I/O subsystem 220 carries the data to memory 225, from which processor 210 retrieves and executes the instructions. The instructions received by memory 225 may optionally be stored on storage 215 either before or after execution by processor 210.

Computer system 205 also includes a communication interface 260 coupled to bus 220. Communication interface 260 provides a two-way data communication coupling to network link(s) 265 that are directly or indirectly connected to at least one communication network, such as a network 270 or a public or private cloud on the Internet. For example, communication interface 260 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 270 broadly represents a local area network (LAN), wide-area network (WAN), campus network, internetwork, or any combination thereof. Communication interface 260 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 260 sends and receives electrical, electromagnetic, or optical signals over signal paths that carry digital data streams representing various types of information.

Network link 265 typically provides electrical, electromagnetic, or optical data communication directly or through at least one network to other data devices, using, for example, satellite, cellular, Wi-Fi, or BLUETOOTH technology. For example, network link 265 may provide a connection through a network 270 to a host computer 250.

Furthermore, network link 265 may provide a connection through network 270 or to other computing devices via internetworking devices and/or computers that are operated by an Internet Service Provider (ISP) 275. ISP 275 provides data communication services through a world-wide packet data communication network represented as internet 280. A server computer 255 may be coupled to internet 280. Server 255 broadly represents any computer, data center, virtual machine, or virtual computing instance with or without a hypervisor, or computer executing a containerized program system such as DOCKER or KUBERNETES. Server 255 may represent an electronic digital service that is implemented using more than one computer or instance and that is accessed and used by transmitting web services requests, uniform resource locator (URL) strings with parameters in HTTP payloads, API calls, app services calls, or other service calls. Computer system 205 and server 255 may form elements of a distributed computing system that includes other computers, a processing cluster, server farm or other organization of computers that cooperate to perform tasks or execute applications or services. Server 255 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 255 may comprise a web application server that hosts a presentation layer, application layer and data storage layer such as a relational database system using structured query language (SQL) or no SQL, an object store, a graph database, a flat file system or other data storage.

Computer system 205 can send messages and receive data and instructions, including program code, through the network(s), network link 265 and communication interface 260. In the Internet example, a server 255 might transmit a requested code for an application program through Internet 280, ISP 275, local network 270 and communication interface 260. The received code may be executed by processor 210 as it is received, and/or stored in storage 215, or other non-volatile storage for later execution.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions.

Several processes may be associated with the same program; for example, opening up several instances of the same program often means more than one process is being executed. Multitasking may be implemented to allow multiple processes to share processor 210. While each processor 210 or core of the processor executes a single task at a time, computer system 205 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

A particular use of the system 200 object of the present invention is disclosed in regard to FIG. 1.

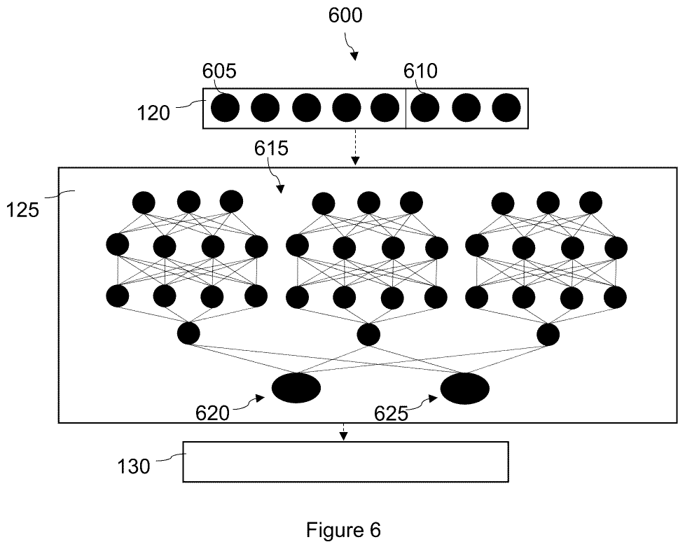

FIG. 4 shows, schematically, a succession of steps of the method 400 object of the present invention. This method 400 to efficiently assemble chemical structures or compositions, comprises:
a step 405 of executing a method such as shown in FIG. 1 and
a step 410 of assembling a chemical structure or composition associated to an output obtained during the step 115 of obtaining.

This step 410 of assembling is configured to materialize the composition. Such a step 410 of assembling may be performed in a variety of ways, such as in a laboratory or a chemical plant for example.

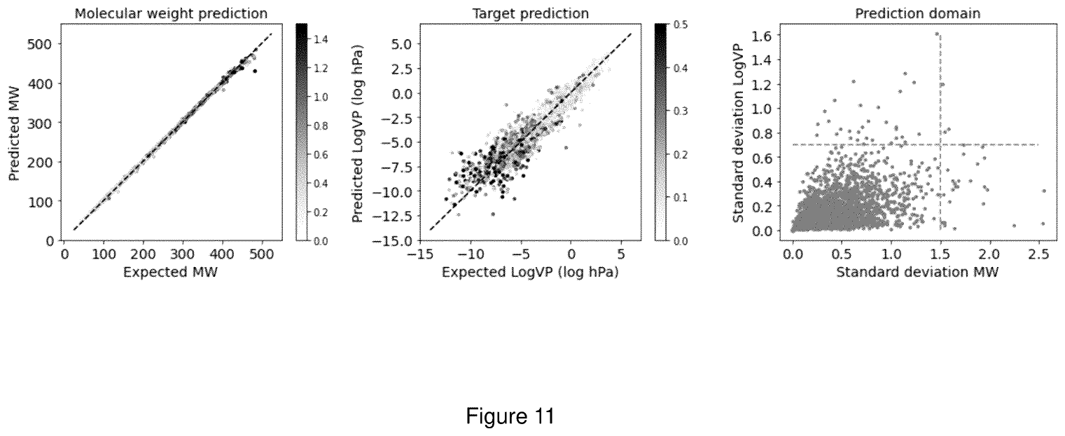

FIG. 8 represents, schematically, a particular implementation example of the method 800 object of the present invention. This method 800 for training an ensemble neural network or multi-branch neural network device is similar to the training performed by the end-to-end ensemble neural network or multi-branch neural network device used in the method 100 object of the present invention.

This method 800 comprises:
a step 805 of inputting the augmented atom and/or bond property hash keys from an exemplar set of chemical structure digital identifiers associated with known outputs that represent at least one physico-chemical and/or odor property associated with an atom and/or bond property hash key,
a step 810 of embedding or tokenizing the input,
a step 815 of operating an ensemble of recursive neural network devices upon the input,
a step 820 of operating an attention layer at the output of the step 815 of operating an ensemble of recursive neural network devices,
a step 825 of operating a flattening layer upon the output of the step 820 of operating an attention layer,
a step 830 of operating a multilayer perceptron (“MLP”) layer upon the output of the flattening layer and
a step 835 of outputting a value for the target odor property and/or physico-chemical property.

The execution parameters of this particular embodiment may be:

| | | Example output |
|---|---|---|
| Layer | Parameters | dimensions |
| Input | Usually, number of horizonal | (N, 10, 21) |
| | augmentations x number of atoms. | |
| Embedding | Number of tokens | (N, 10, 21, 40) |
| RNN | Cell type, bidirectionality, sequence | (N, 10, 21, 20) |
| | length | |
| Attention | Selected dimension | (N, 21, 20) |
| Flatten | — | (N, 420) |
| MLP | Variable set of one or more fully | (N, 30) |
| | connected layers, optionally with a | |
| | selected activation function and | |
| | dropout. | |
| Y | Number of targets plus activation | (N, 1) + linear |
| | | activation |
| | | (N, 4) + softmax |
| | | activation |

Total parameters: 52,166

In this table, the number N represents the number of points (e.g., input batch size). In this architecture a chemical structure is displayed as an augmented 20-chip, which is subsequently converted using an embedding layer and recursive neural network layer. The attention layer runs a feature selection. The MLP part of the network is a fully connected neural network with activation.

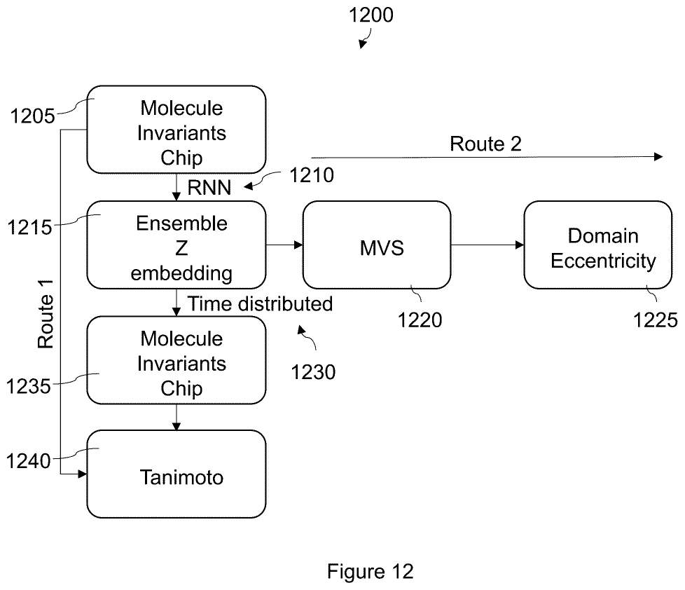

FIGS. 9 to 11 show performance of the architecture shown in FIG. 8 relative to three distinct targets:

FIG. 9 shows the performance for odor detection threshold (“QODT”),

FIG. 10 shows the performance for volatility and

FIG. 11 shows the performance for Log VP (that is the logarithmic form of the vapor pressure).

As it is understood, the present invention also aims at a computer implemented ensemble neural network or multi-branch neural network device, in which the ensemble neural network or multi-branch neural network device is obtained by any variation of the computer-implemented method 300 object of the present invention.

As it is understood, the present invention also aims at a computer program product, comprising instructions to execute the steps of a method 300 object of the present invention when executed upon a computer.

As it is understood, the present invention also aims at a computer-readable medium, storing instructions to execute the steps of a method 300 object of the present invention when executed upon a computer.

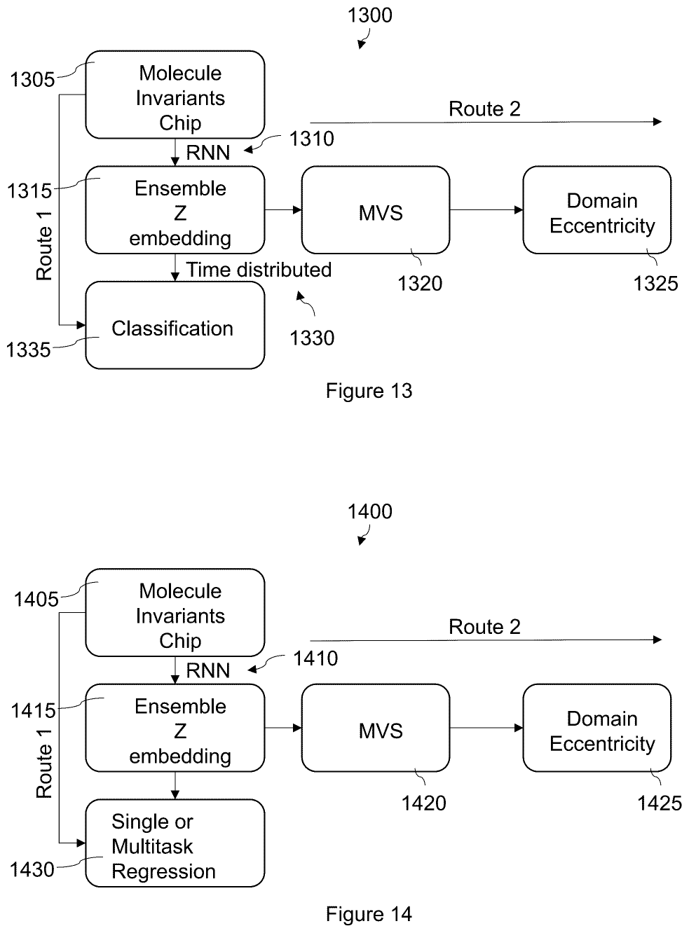

FIG. 12 shows, schematically, a training architecture 1200 to select, from a set of chemical structures, chemical structures that provide a particular feature, such as an insect repellent capacity value above a determined threshold.

Such an architecture 1200 comprises:
as an input 1205, a hash vectors of at least one atomic property in a chemical structure, and at least one set of outputs, said outputs corresponding to odor properties associated with the set of inputs,
an ensemble neural network or multi-branch neural network 1210 comprising a set of recursive neural networks to generate an embedding 1215,
two alternative and compatible routes may then be implemented:
   in a first route, a multivariant statistic algorithm 1220 can be used, complemented with a numerical domain eccentricity evaluation algorithm 1225,
   in a second route, a time distribution 1230 of the embedding is performed, to obtain an alternative input 1235, complemented with the use of an ensemble 1240 neural network using Tanimoto neural networks, such as disclosed in the present invention.

FIG. 13 shows, schematically, a training architecture 1300 to classify, from a set of chemical structures, chemical structures that provide a particular feature, such as a biodegradability value.

Such an architecture 1300 comprises:
as an input 1305, a hash vectors of at least one atomic property in a chemical structure, and at least one set of outputs, said outputs corresponding to odor properties associated with the set of inputs,
an ensemble neural network or multi-branch neural network 1310 comprising a set of recursive neural networks to generate an embedding 1315,
two alternative and compatible routes may then be implemented:
   in a first route, a multivariant statistic algorithm 1320 can be used, complemented with a numerical domain eccentricity evaluation algorithm 1325,
   in a second route, a time distribution 1330 of the embedding is performed, complemented with the use of an ensemble 1335 neural network using a classification neural network, such as disclosed in the present invention.

FIG. 14 shows, schematically, a training architecture 1400 to predict, for a set of chemical structures, values for a particular feature, such as an odor detection threshold value.

Such an architecture 1400 comprises:
as an input 1405, a hash vectors of at least one atomic property in a chemical structure, and at least one set of outputs, said outputs corresponding to odor properties associated with the set of inputs,
an ensemble neural network or multi-branch neural network 1410 comprising a set of recursive neural networks to generate an embedding 1415,
two alternative and compatible routes may then be implemented:
   in a first route, a multivariant statistic algorithm 1420 can be used, complemented with a numerical domain eccentricity evaluation algorithm 1425,
   in a second route, an ensemble 1430 neural network using single or multitask regression neural networks, such as disclosed in the present invention.

As it can be understood, the present invention may be used to act as a filtration technique, using any predicted physico-chemical property and/or odor property to label molecule or ingredient digital identifiers in a database, said molecules or ingredients being selected as worthwhile points of exploration by flavorists and perfumers.

As it can be understood, the present invention may be used with, as inputs, couples of molecules to predict the proximity of molecules in the couple or by using the difference observed in the couple for regression or classification.

As it can be understood, the present invention may be used as a classifier used in relation to physico-chemical and/or odor property values for a chemical structures or compositions.

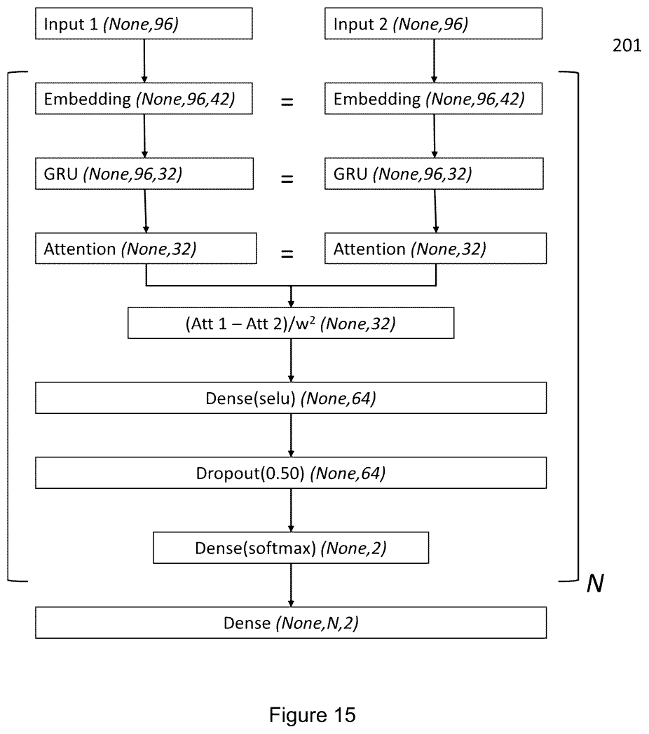

FIG. 15 shows a particular architecture which highlights the performance of such a classifier.

In machine learning, chi-squared testing is often used to evaluate the performance of classification models. For example, suppose one have a binary classification problem where one want to predict whether a patient has a disease or not. one can use a chi-squared test to determine if our model is performing better than chance by comparing the predicted class distribution to the expected class distribution.

In ensemble learning, where multiple models are combined to improve overall performance, chi-squared testing can be used to evaluate the performance of the ensemble. Ensemble learning is a popular technique in machine learning where multiple models are trained and combined to improve overall performance. By using multiple models, one can reduce the risk of overfitting and improve the robustness of the model.

In an ensemble of classification models, each model makes an independent prediction on the input data, and the final prediction is made by combining the predictions of all models. Chi-squared testing can be used to evaluate the performance of the ensemble by comparing the predicted class distribution of the ensemble to the expected class distribution. If the ensemble is performing better than any individual model, one can conclude that the ensemble is effective.

Overall, chi-squared testing is a powerful tool for evaluating the performance of machine learning models and ensembles. By using chi-squared testing, one can make informed decisions about which models to use and how to improve them.

Forced-choice modeling is an example of a contrastive classification task, where the goal is to identify the correct example from a set of alternatives. This type of task is commonly encountered in many real-world scenarios, such as identifying the correct answer in a multiple-choice exam or recognizing a specific object from a set of similar objects. In science, results are frequently evaluated in a relative setting by comparing two or more candidates between each other. one therefore hypothesize that contrastive neural networks, trained to select the more promising entry from a set of alternatives may provide valuable models.

By making pairs or triplets or other alternates in a contrastive neural network, data can be augmented. Indeed, for a regression task one can augment the data from N to N²—N pairs, or to (N²—N)/2 pairs, considering only the lower-half or upper-half matrix. Alternately, in problems with small hit rates, hits can be coupled with one or more non-hits in a forced-choice classification. In the latter experiment the model is trained to detect the hit molecule from the proposed options. Another benefit of these contrastive networks includes the creation of balanced sets. one may indeed expect that the lower values are evenly distributed on the number of alternates.

To tackle this task, one can use an ensemble neural network with individual votes, where each model in the ensemble makes an independent prediction on the input data. The final prediction is then made by combining the predictions of all models. By using an ensemble of models, one can reduce the risk of overfitting and improve the robustness of the model.

After making the prediction, one can use chi-squared testing to measure the statistical significance of the decision. In this case, one can compare the predicted class distribution to the expected class distribution, which is a uniform distribution over the three examples. If the chi-squared test shows that the predicted class distribution is significantly different from the expected class distribution, one can conclude that the ensemble is performing well and is able to correctly identify the correct example from the input X.

Overall, using an ensemble neural network with individual votes and chi-squared testing is a powerful approach for contrastive classification tasks, and can help improve the accuracy and robustness of the model. By using this approach, one can make informed decisions about which examples are correct and which are not and improve our ability to recognize and classify objects in real-world scenarios.

To perform such calculations, one can use SMILES strings that contain explicit-implicit hydrogen atoms. For instance, let us consider the molecule toluene. The explicit SMILES for toluene, which is written as “[CH3][c]1[cH][cH][cH][cH][cH]1”, can be tokenized by grouping the atoms defined by characters enclosed in square brackets, from [to]. All other characters, such as the ring index 1, can be tokenized as individual characters. Therefore, the tokenized SMILES for toluene is “[CH3] [c]1 [cH] [cH] [cH] [cH] [cH] 1”. Similarly, the explicit SMILES for glutamic acid, which is “[NH2][CH]([CH2][CH2][C](═[O])[OH])[C](═[O])[OH]”, can be tokenized by individually tokenizing the bonds, such as ═ for a double bond, and branches, i.e., (and). The tokenized SMILES for glutamic acid is “[NH2] [CH] ([CH2] [CH2] [C] (=[0]) [OH]) [C] (=[0]) [OH]”.

The forced-choice classification is run using a network layout where the same embedding, GRU and attention and latent layer are applied to all input entries, followed by the creation of a learnable contrastive layer, creating the differences between all pairs.

FIG. 15 shows an architecture layout of a contrastive classifier, asked to select the molecule with the lowest molecular weight. The input is a tokenized vector with integer-based tokens, followed by a Keras' Embedding layer, a Keras' GRU layer and an attention layer. The equal sign between these layers indicate that the same layers are applied to both entries. A trainable contrastive layer creates the difference between the output of attention 1 and attention 2. A multi-layer perceptron with dropout is used for the classification task. The model is repeated N times to create an ensemble neural network. The values (None,x) and (None,x,y) indicate the output shape of the layers.

Such a contrastive classifier can be trained using a dataset obtained from NIST. The data can be split into a training set of 8,518 molecules, a validation set of 819 molecules and a test set of 772 molecules. To follow the on-training performance on every epoch a train and validation dataset can be used. To train the network, 45,458 pairs of molecules can be built with a maximum difference of 14.02 g/mol, which corresponds to the mass of one CH2 group in a molecule. The classifier can be trained to detect the molecule with the highest molecular weight. Note that any numerical target can be trained, including linear retention index, volatility, or odor-detection threshold. The validation set can contain a number of pairs with a maximum difference of 14.02 g/mol between the molecules. Every epoch can be trained using 46 iterations with a batch-size of 1000 pairs per iteration. The model is trained using mean binary-crossentropy computed over all models in the ensemble.

On completion the performance can be tested on a test set, composed of a number of pairs with a maximum difference of 14.02 g/mol between the molecules. The results on the performance are displayed in table 1, below. Given that the model performs a relative classification task and one have asked to identify the position of the lowest molecular weight, the results are only the accuracy is reported. In table 1, the p-value is computed using chi-squared testing on the votes produced by the ensemble. The result is considered conclusive if the p-value for the vote proportions dropped below 0.05. From the of table 1, one can clearly see that the results for the conclusive results are clearly better than the decision on the non-conclusive entries.

TABLE 1

| | Number | Accuracy |
|---|---|---|
| All data | 4554 (100%) | 98.0 +/− 0.2% |
| Conclusive p < 0.05 | 4227 (92.8%) | 99.8 +/− 0.1% |
| Inconclusive p > 0.05 | 327 (8.4%) | 74.9 +/− 2.4% |

To sum up, using ensemble models for classification significantly boosts the capacity to convey the confidentiality of the outcomes. The forecast is formed by a combination of multiple votes on the class, along with an indication of the level of confidence in the prediction (table 2).

TABLE 2

| Decision | True | False |
|---|---|---|
| Conclusive p < 0.05 | Validated data point. | Data points need to be |
| | | reviewed. |

| | |
|---|---|
| Inconclusive p > 0.05 | Votes tie in model: no reliable prediction. |

In conclusion, the methodology displayed can be used for both relative and absolute classification tasks. The example above shows a relative task to learn to select the molecule with the higher molecular weight. In such classification, a regression task is converted into a contrastive classification task. In an absolute classifier, an ensemble is asked to predict the class defined in the data, such as performed in the MNIST dataset to detect numbers in an image.

1. Method to predict at least one physico-chemical and/or odor property value for a chemical structure or composition, comprising the steps of:
defining, upon a computer interface, a digitized representation of a chemical structure or composition,
executing, by a computing device, upon the digitized representation defined, an end-to-end trained ensemble neural network or multi-branch neural network model to predict at least one physico-chemical and/or odor property value for a chemical structure or composition,
providing, upon a computer interface, the at least one physico-chemical and/or odor property value for a chemical structure or composition,
further comprising the steps of:
providing a set of exemplar data, comprising at least one set of inputs, said inputs corresponding to digitized representations of chemical structures or compositions, and at least one set of outputs, said outputs corresponding to physico-chemical and/or odor properties associated with the set of inputs, to an end-to-end ensemble neural network or multi-branch neural network device comprising:
several neural network sub-devices, each sub-device being configured to provide an independent prediction based upon the exemplar data,
a layer configured to output at least one value based on, or representative of, the distribution of said independent predictions and
said layer comprising a sampling device configured to output at least one random value as a function of a probability distribution representative of the distribution of independent predictions, said output random values being computed in a differentiable way and used for backpropagation within the end-to-end ensemble neural network or multi-branch neural network device,
operating the end-to-end ensemble neural network or multi-branch neural network device based upon the set of exemplar data and
obtaining the trained end-to-end ensemble neural network or multi-branch neural network model configured to predict physico-chemical and/or odor properties for input digitized representations of chemical structures or compositions.

2. Method according to claim 1, in which at least one set of inputs of the exemplar data corresponds to hash vectors of at least one atomic property in a chemical structure or composition, the method further comprising, upstream of the step of executing, a step of converting the defined digitized chemical structure or composition into a set of hash vectors of at least one atomic property representative of the digitized chemical structure or composition, said set of hash vectors being used as input during the step of executing.

3. Method according to claim 2, in which at least one hash vector of an atomic property is representative of one of the following:
atomic number of the corresponding atom,
atomic symbol of the corresponding atom,
mass of the atom,
explicit map number,
row index in the periodic system,
column index in the periodic system,
total number of hydrogens on the atom,
implicit number of hydrogens on the atom,
explicit number of hydrogens on the atom,
degree of the atom,
total degree of the atom,
the valence state of the atom,
the implicit valence of the atom,
the explicit valence of the atom,
formal charge on the atom,
partial charge on the atom,
electronegativity on the atom,
number of bonds by bond type,
number of neighbors by atomic number, wild card,
number of neighbors by bond type plus atomic number, wild card,
number of neighbors by wild card,
value to indicate aromaticity,
value to indicate aliphatic atom,
value to indicate a conjugated atom,
value to indicate cyclic atom,
value to indicate a macrocyclic atom,
value to indicate a geometrically constraint atom,
value to indicate electron withdrawing atom,
value to indicate an electron donating atom,
value to indicate the reaction site,
value to indicate hydrogen bond donor,
value to indicate hydrogen acceptor,
value to indicate the multivalence as hydrogen bond donor,
value to indicate the multivalence as hydrogen bond acceptor,
number of cycles on the atom,
ring size on the atom,
hybridization state of the atom,
values to indicate the atomic geometry,
number of electrons in the atomic orbitals,
number of electrons in lone pairs,
radical state,
the isotope on the atom,
atom center symmetric functions,
value for relative stereochemistry as chi clockwise, chi anticlockwise,
value for absolute stereochemistry,
value for absolute stereochemistry,
value for double bond stereochemistry,
value for priority for determination of stereochemistry,
value representative of a positive or negative impact of an atom upon a determined training target, to indicate knowledge-based enrichment contribution,
value representative of a positive or negative impact of an atom upon a determined training target, to indicate knowledge-based dilution contribution and/or
value for ring stereochemistry.

4. Method according to claim 2, in which at least one hash vector of a bond property is representative of one of the following:
bond order,
bond type,
stereochemistry of the bond:
bond direction for tetrahedral stereochemistry,
bond direction for double bond stereochemistry or
bond direction for spatial orientation,
atomic number(s) for the “from” and/or “to” atoms,
atomic symbols for the “from” and/or “to” atoms,
dipole moment in the bond,
quantum-chemical properties:
electron density in the bond,
electron configuration of the bond,
bond orbitals,
bond energies,
attractive forces,
repulsive forces,
bond distance,
aromatic bond,
aliphatic bond,
ring properties of the bond:
number of rings on the bond,
ring size(s) of the bond,
smallest ring size of the bond,
largest ring size of the bond,
rotatable bond,
spatially constrained bond,
hydrogen bonding properties,
ionic bonding properties,
bond order for reactions, including the “null” bond to identify a broken/formed bond in a reaction:
bond order in reagents,
bond order in intermediate products or
bond order in transition states.

5. Method according to claim 1, in which at least one output value representative of the distribution is representative of a dispersion of the distribution.

6. Method according to claim 5, in which the end-to-end ensemble neural network or multi-branch neural network device is trained to minimize at least one value representative of the dispersion of the distribution.

7. Method according to claim 1, in which at least one odor property is representative of:
an insect repellent capability value,
a sensory property value,
a biodegradability value,
an antibacterial value,
an odor detection threshold value,
an odor strength value,
a top-heart-base value,
a hazard value,
a biological activity for taste
a biological activity for olfaction,
a biological enhancing or modulating taste activity,
a biological enhancing or modulating olfaction activity and/or
an olfactive smell description.

8. Method according to claim 1, in which at least one physical property is representative of:
a boiling point value,
a melting point value,
a water solubility value,
a Henry constant value,
a vapor pressure value,
a volatility value or
a headspace concentration value.

9. Method according to claim 1, in which at least one neural network device is:
a recursive neural network device,
a graph neural network device,
a variational autoencoder neural network device or
an autoencoder neural network device.

10. Method according to claim 9, which comprises, upstream of the step of providing input data to and end-to-end ensemble neural network or multi-branch neural network device, a step of atom or bond relationship vector augmentation.

11. Method according to claim 10, in which the step of atom or bond relationship vector augmentation comprises a step of horizontal augmentation, configured to provide several vectors representing a single digitized representation of a molecular structure or composition, each vector representing a particular representation of the canonical representation molecular structure or composition, each vector being treated as a single input during the step of providing.

12. Method according to claim 11, in which the step of atom or bond relationship vector augmentation comprises a step of vertical augmentation, to create several groups of several horizontal augmentations, representing a unique molecular structure or composition, each group being treated as a single input during the step of providing.

13. Method to efficiently assemble chemical structures or compositions, comprising:
a step of executing a method according to claim 1, and
a step of assembling a chemical structure or composition associated to an output obtained during the step of obtaining.

14. System to predict at least one physico-chemical and/or odor property value for a chemical structure or composition, comprising the means of:
defining, upon a computer interface, a digitized representation of a chemical structure or composition,
executing, by a computing device, upon the digitized representation defined, an end-to-end trained ensemble neural network or multi-branch neural network model to predict at least one physico-chemical and/or odor property value for a chemical structure or composition,
providing, upon a computer interface, the at least one physico-chemical and/or odor property value for a chemical structure or composition,
further comprising the means of:
providing a set of exemplar data, comprising at least one set of inputs, said inputs corresponding to digitized representations of chemical structures or compositions, and at least one set of outputs, said outputs corresponding to physico-chemical and/or odor properties associated with the set of inputs, to an end-to-end ensemble neural network or multi-branch neural network device comprising:
several neural network sub-devices, each sub-device being configured to provide an independent prediction based upon the exemplar data,
a layer configured to output at least one value based on, or representative of, the distribution of said independent predictions and
said layer comprising a sampling device configured to output at least one random value as a function of a probability distribution representative of the distribution of independent predictions, said output random values being computed in a differentiable way and used for backpropagation within the end-to-end ensemble neural network or multi-branch neural network device,
operating the end-to-end ensemble neural network or multi-branch neural network device based upon the set of exemplar data and
obtaining the trained end-to-end ensemble neural network or multi-branch neural network model configured to predict physico-chemical and/or odor properties for input digitized representations of chemical structures or compositions.
